(19) United States
(12) Patent Application Publication
(10) Pub. No.: US 2003/0148359 A1
(43) Pub. Date: Aug. 7, 2003

(54) Saxitoxin detection and assay method

(75) Inventors: Edward Moczydlowski, Madison, CT (US); Gomathi Krishnan, Somerville, MA (US); Maria A. Morabito, Boston, MA (US)

Correspondence Address: ROPES & GRAY LLP, ONE INTERNATIONAL PLACE, BOSTON, MA, 02110-2624, US

(73) Assignee: Yale University, New Haven, CT (US)

(21) Appl. No.: 10/342,103

(22) Filed: Jan. 13, 2003

Related U.S. Application Data
(60) Provisional application No. 60/348,086, filed on Jan. 11, 2002.

(51) Int. Cl. C12Q 1/68; G01N 33/53; C07H 21/04; C07D 487/14; C12P 21/02; C12N 5/06; C07K 14/705

(52) U.S. Cl. 435/6; 435/7.1; 435/69.1; 435/320.1; 435/325; 530/350; 536/23.5; 544/251

(57) ABSTRACT

Tagged saxiphilin and tagged fragments thereof that bind saxitoxin and saxitoxin derivatives, methods of producing tagged saxiphilin and tagged fragments and methods of detecting and measuring saxitoxin and saxitoxin derivatives in samples.

### RELATED APPLICATION

This application claims the benefit of the filing date of U.S. Provisional Application No. 60/348,086, entitled “Flag-epitope-and hexahistidine-tagged derivatives of saxiphilin for use in the detection and assay of saxitoxin”, by Edward G. Moczydlowski, Gomathi Krishnan and Maria Morabito (filed Jan. 11, 2002). The entire teachings of the referenced Provisional Application are incorporated herein by reference.

### FUNDING

Work described herein was funded, in whole or in part, by National Institutes of Health grant GM51172. The United States government has certain rights in the invention.

### BACKGROUND OF THE INVENTION

Saxiphilin is a soluble protein that binds saxitoxin (STX) with high affinity and specificity (Mahar, et al., 1991). STX and STX derivatives are microbial neurotoxins responsible for paralytic shellfish poisoning, which results from block of voltage-sensitive sodium channels by these toxins (Shimizu, 1996). Saxiphilin was first discovered in frogs (Doyle, et al., 1982; Moczydlowski, et al., 1988), but similar soluble STX binding activity has been observed in diverse species of arthropods, amphibians, reptiles, and fish (Llewellyn, et al. 1997). Paralytic shellfish poisoning is a health problem associated with toxic algal blooms and resultant shellfish contamination in coastal areas around the world (Anderson, 1989; Baden, 1988). Toxicity of shellfish extracts has been commonly determined by live mouse bioassay (Schantz, et al., 1958; Sommer and Meyer, 1937). Various in vitro assays have been developed for the screening and analysis of STX and STX derivatives in shellfish extracts based on HPLC, fluorescence detection, immunochemical detection, binding to sodium channels, and binding to saxiphilin (Doucette et al., 1997; Gessner, et al., 1997; Indrasena and Gill, 1998; Kralovec, et al., 1996; Llewellyn, et al., 1998; Negri and Llewellyn 1998, WO 02/48671. These references are hereby incorporated by reference in their entirety). It would be helpful to have additional approaches for assessing toxicity of shellfish, particularly those which are highly sensitive/exhibit great specificity for saxitoxin.

### SUMMARY OF THE INVENTION

The present invention relates to tagged saxiphilin and tagged saxiphilin fragments; nucleic acids (DNA, RNA) that encode tagged saxiphilin or tagged saxiphilin fragments (referred to collectively as tagged saxiphilin-encoding nucleic acids, e.g., tagged saxiphilin-encoding DNA or RNA); vectors (e.g., baculovirus vectors) comprising tagged saxiphilin-encoding nucleic acid that are expressed in appropriate host cells to produce tagged saxiphilin or tagged saxiphilin fragments; and host cells, such as insect cells, in which tagged saxiphilin and/or tagged saxiphilin fragments are expressed from vectors of the present invention. The invention further relates to use of tagged saxiphilin and tagged saxiphilin fragments, such as in methods of assessing samples for the occurrence (presence or absence) of paralytic shellfish toxin (e.g., saxitoxin); methods of detecting and/or quantifying (determining the amount of) paralytic shellfish toxin, such as saxitoxin and saxitoxin derivatives, in samples and methods of isolating saxitoxin and saxitoxin derivatives from a sample. Such methods are also the subject of this invention. Samples to which methods of the present invention can be applied include biological samples, such as seafood and water; other biological samples (e.g., blood, urine, saliva); and soil (e.g., from shellfish beds). The present invention also relates to methods of purifying or isolating tagged saxiphilin or tagged saxiphilin fragments and methods of purifying or isolating saxitoxins from sources in which they occur (e.g., cells in which tagged saxiphilin is expressed, biological samples contaminated with saxitoxin). Kits useful for assessing samples for the occurrence or quantity of saxitoxin are also the subject of this invention.

The present invention provides reagents and methods useful in detecting and quantifying saxitoxin and saxitoxin derivatives and, thus, in addressing a public health problem.

### BRIEF DESCRIPTION OF THE DRAWINGS

FIGS. 1A and 1B show a scheme for construction of Flag-His₆-tagged versions of saxiphilin and C-lobe saxiphilin. FIG. 1A shows the sequence of Sax SmaI sense oligonucleotide primer (SEQ ID NO: 1), the encoded amino acid sequence (SEQ ID NO: 2) and the Sax Flag-His6 antisense primer (SEQ ID NO: 3). It also shows the amino acid sequence (SEQ ID NO: 4) encoded by the sense strand (the complement), 5′CT GCC TGC ACA ATT AAA CAC TGT GCA GAC TAC AAG GAT GAC GAT GAC AAG ATC GTG GGA GGT CAT CAT CAT CAT CAT CAT TAA GGT ACC ATG GTC GAC TTA C 3′(SEQ ID NO: 5) of the nucleic acid sequence, 3′ GA CGG ACG TGT TAA TTT GTG ACA CGT CTG ATG TTC CTA CTG CTA CTG TTC TAG CAC CCT CCA GTA GTA GTA GTA GTA GTA ATT CCA TGG TAC CAG CTG AAT G 5′ (SEQ ID NO: 6). The underlined portions of SEQ ID NO: 4 are, respectively, the amino acid sequence of the Flag epitope (SEQ ID NO: 7) and the hexahistidine sequence (SEQ ID NO: 9). An asterisk (*) marks the location of the original C-terminal Cys residue of saxiphilin. FIG. 1B is a schematic of the restriction digestion of saxiphilin R-Sax or C-Sax cDNA and the Flag-His₆-tagged PCR product followed by ligation of the selected products into the pBluebac4 vector.

FIG. 2 is a graphic representation showing results of Ni²⁺-chelate affinity chromatography of R-Sax-FH₆ on Ni²⁺-NTA resin. A sample of insect cell medium containing R-Sax-FH₆ was loaded on a column (2.0×15 cm) of Ni²⁺-NTA resin. The column was washed with buffer containing 30% glycerol and 4 mM imidazole. R-SAX-FH₆ was eluted from the resin with 150 mM imidazole buffer. Fractions (4 ml) were assayed for [³H]STX binding and protein absorbance at 280 nm.

FIGS. 3A and 3B are graphic representations of results of binding titration of pure R-Sax-FH₆ (FIG. 3A) and semi-pure C-Sax-FH₆ (FIG. 3B) with [³H]STX. Plots show raw binding data with non-specific background subtracted. Insets represent the corresponding Scatchard plots. Data points are the mean of duplicate samples assayed in the absence and presence of 10 μM unlabeled STX for background subtraction. For the Scatchard plots the K_{D} was 0.75 nM for R-Sax-FH₆ and 2.7 nM for C-Sax-FH₆.

FIGS. 4A and 4B are graphic representations showing association and dissociation kinetics of [³H]STX binding to pure R-Sax-FH₆ (FIG. 4A) and semi-pure C-Sax-FH₆ (FIG. 4B). Association rate data (o) measured in the presence of 10 nM [³H]STX were normalized to the equilibrium level of binding and fit using a pseudo first-order rate constant of 0.0082 s⁻¹ for R-SAX-FH₆ and 0.0095 s⁻¹ for C-SAX-FH₆, (top horizontal axis). Dissociation rate data (●) were normalized to [³H]STX bound before the addition of excess unlabeled STX and fit to a first-order rate constant of 1.68×10⁻⁴ s⁻¹ for R-Sax-FH₆ and 3.7×10⁻⁴ s⁻¹ for C-SAX-FH₆ (bottom horizontal axis).

FIGS. 5A and 5B are graphic representations showing results of solid Phase assay of R-Sax-FH₆ and displacement of [³H]STX binding to Reacti-bind™ metal chelate plates by unlabeled STX. FIG. 5A: 5 nM [³H]STX in a 200 μl reaction mixture containing 25 mM Hepes-NaOH, 150 mM NaCl, pH 8.0, was titrated with increasing amounts of R-Sax-FH₆ in Reacti-bind™ metal chelate wells and incubated on ice for a period of 3 hours. [³H]STX bound to the wells was counted after washing the wells 3 times with ice-cold 100 mM Tris-HCl buffer, pH 8. FIG. 5B: Competition of [³H]STX binding to R-Sax-FH₆ in Reacti-bind™ metal chelate plate wells by unlabeled STX. A fixed amount of R-Sax-FH₆ with 5 nM [³H]STX in the standard incubation mixture was titrated with an increasing concentration of unlabeled STX.

FIGS. 6A-6D are the nucleic acid and amino acid sequences of full length bullfrog saxiphilin tagged at the C-terminus with a Flag epitope and a hexahistidine sequence (R-Sax-FH₆) and C-lobe saxiphilin tagged at the C-terminus with a Flag epitope and a hexahistidine sequence (C-Sax-FH₆).

FIG. 6A is the nucleic acid sequence of full-length bullfrog saxiphilin tagged at the C-terminus with a Flag epitope and a hexahistidine sequence (SEQ. ID NO: 11).

FIG. 6B is the amino acid sequence of full-length bullfrog saxiphilin tagged at the C-terminus with a Flag epitope and a hexahistidine sequence (SEQ ID NO: 12)

FIG. 6C is the nucleic acid sequence of C-lobe bullfrog saxiphilin tagged at the C-terminus with a Flag epitope and a hexahistidine sequence (SEQ ID. NO: 13)

FIG. 6D is the amino acid sequence of C-lobe bullfrog saxiphilin tagged at the C-terminus with a Flag epitope and a hexahistidine sequence (SEQ ID NO: 14)

### DETAILED DESCRIPTION OF THE INVENTION

Described herein are tagged saxiphilin and fragments thereof (tagged saxiphilin fragments) that bind saxitoxin and/or saxitoxin derivatives; isolated nucleic acid (e.g., DNA, RNA) encoding tagged saxiphilin and fragments thereof that bind saxitoxin and/or saxitoxin derivatives, methods of producing and, optionally, isolating, tagged saxiphilin and tagged saxiphilin fragments; methods in which tagged saxiphilin and tagged saxiphilin fragments are used, and kits containing tagged saxiphilin and tagged saxiphilin fragments. As used herein, the term “saxitoxin derivatives” includes compounds also referred to as paralytic shellfish poisoning (PSP) toxins, such as but not limited to: N1-hydroxylated derivatives of STX (e.g., NEO, GTX-I, GTX-IV, B2, C3, C4), C11-sulfated derivatives of STX (e.g., GTX-I, GTX-II, GTX-III, GTX-IV, C3 C1, C2, C4), the sulfamate toxins (e.g., B1, B2, C3, C1, C2, C4), and the decarbamoyl derivatives (e.g., dc-STX, dc-NEO, dc-GTX-I, dc-GTX-II, dc-GTX-III, dc-GTX-IV) (Sullivan, et al, 1988).

Tagged saxiphilin and tagged saxiphilin fragments of the present invention comprise all or a portion of the amino acid residues of a saxiphilin of invertebrate or vertebrate origin and an (one or more) affinity tag, which is a series of amino acid residues (e.g., an oligopeptide) not present in saxiphilin as it occurs in nature. Amino acid residues are represented herein by their single letter designations. As used herein, the term “saxiphilin derived from” includes saxiphilin protein isolated from a source in which it occurs in nature (e.g., bullfrog) and saxiphilin protein which has substantially the same amino acid sequence and/or function (e.g., ability to bind saxitoxin and/or saxitoxin derivatives) as naturally occurring saxiphilin and has been produced by techniques such as recombinant DNA or RNA techniques or chemical or other synthetic methods. Amino acid substitutions which maintain substantially the same properties (e.g., charge specificity, hydrophobicity, polarity, structure) of the respective amino acid residues of naturally occurring saxiphilin protein are also included. Similarly, the term “DNA derived from” includes DNA obtained from a source in which it occurs in nature and DNA produced by recombinant DNA technology or chemical synthetic methods which encodes substantially the same amino acid sequence as naturally occurring saxiphilin. The term “DNA encoding saxiphilin or saxiphilin fragments” includes DNA whose sequences are presented herein and additional sequences which, due to the degeneracy of the genetic code, encode saxiphilin, such as saxiphilin nucleic acid (SEQ ID NO: 15) and amino acid (SEQ ID NO: 16) sequences as provided in GenBank Accession number U05246. (Morabito, M A and Moczydlowksi, E, 1994a and 1994b). The present invention also comprises isolated nucleic acid encoding all or a portion of tagged saxiphilin or a tagged fragment thereof, e.g., nucleic acid encoding saxiphilin or a fragment thereof of vertebrate or invertebrate origin and an affinity tag.

A wide variety of tags can be present on a tagged saxiphilin or tagged saxiphilin fragment of the present invention. For example, a Flag epitope, hexahistidine sequence, a streptavidin sequence (Strep-tag), calmodulin binding peptide (CBP), maltose binding protein (MBP), cellulose binding domain (CBD) tag, myc epitope, hemmaglutinin (HA) tag, glutathione-S-transferase (GST) fusion, or thioredoxin fusion, individually or in combination (one or more can be present on saxiphilin or a saxiphilin fragment).

For example, saxiphilin or a fragment thereof (e.g., the C-lobe of saxiphilin) tagged with a (one or more) Flag epitope (DYKDDDDK; SEQ ID NO:7); a (one or more) hexahistidine (HHHHHH; SEQ ID NO: 9) sequence; or a (one or more) Flag epitope and a (one or more) hexahistidine sequence can be produced and used in methods of assessing or analyzing samples for saxitoxin (STX) and saxitoxin derivatives. Bullfrog saxiphilin binds saxitoxin and saxitoxin derivatives with high affinity and 1:1 stoichiometry and, thus, is particularly useful in methods of the present invention. In specific embodiments, the invention is bullfrog saxiphilin tagged with a Flag epitope, a hexahistidine sequence or both (at least one Flag epitope and at least one hexahistidine sequence) or tagged fragments of bullfrog saxiphilin that bind saxitoxin and/or saxitoxin derivatives. For example, the C-lobe of bullfrog saxiphilin tagged with at least one Flag epitope, at least one hexahistidine sequence, or both is one embodiment of the present invention. In specific embodiments, the invention relates to tagged bullfrog saxiphilin in which the tag is a Flag epitope and a hexahistidine sequence and tagged bullfrog C-lobe of saxiphilin in which the tag is a Flag epitope and a hexahistidine sequence.

Tags are typically placed at the C-terminus of saxiphilin or a fragment thereof, although they may be present in any location (e.g., N-terminus, internally) on the saxiphilin protein or fragment, provided that the resulting tagged product undergoes proper folding and, preferably, is secreted from cells in which it is produced. Such tags can be continuous with the saxiphilin protein or can be separated from the saxiphilin protein by intervening amino acid residues, which are introduced, for example, in order to permit optimal binding to the tags and/or to introduce proteolytic or chemical cleavage sites for specific removal of the tags. For example, an additional intervening amino acid residue can be present between saxiphilin and tag sequences (e.g., as described in the examples) or more than one additional intervening amino acid residue can be included. In the embodiments in which tagged saxiphilin comprises at least one Flag epitope and at least one hexahistidine sequence, the two tags will typically be in tandem (with no intervening amino acid residues) or separated by a limited number of amino acid residues (e.g., 1-5 residues). For example, if a tagged saxiphilin comprises a Flag epitope and a hexahistidine sequence, the two tags can be continuous (with no intervening amino acid residues) or can be separated by additional amino acid residues (e.g., as described in the example). In a specific embodiment of the present invention, 19 amino acid residues (ADYKDDDDKIVGGHHHHHH; SEQ ID NO: 17) are present on a tagged saxiphilin or a fragment thereof: DYKDDDDK (SEQ ID NO: 7) as the Flag epitope; HHHHHH (SEQ ID NO: 9) as the hexahistidine sequence; A between the C-terminus of saxiphilin and the initial amino acid residue (D) of the Flag tag; and IVGG linking the Flag tag and the hexahistidine sequence. In particular embodiments, the 19 amino acid residues are present at the C-terminus of bullfrog saxiphilin and at the C-terminus of the C-lobe of bullfrog saxiphilin. Other amino acid residues can be used as linkers between a saxiphilin amino acid residue (e.g., the amino acid residue at the saxiphilin C-terminus) and a tag amino acid residue (e.g., the initial D of the Flag epitope or initial H of a hexahistidine sequence). Other types of useful linkers would be polyamino acid sequences of residues that do not interfere with functions or biochemical applications of the protein such as: polyA (e.g., AAA), polyG (e.g., GGG), or polyQ (e.g., QQQ) sequences.

Tagged saxiphilins of any origin (e.g., invertebrate, such as an arthropod, including centipedes, isopods, spiders, crabs and insects, or vertebrate, such as bullfrog and other amphibians, reptiles and fish) and tagged fragments thereof that bind saxitoxin and saxitoxin derivatives are also embodiments of the present invention.

The presence of such tags facilitates biochemical manipulation and production of a solid-phase matrix that binds saxitoxin and saxitoxin derivatives. Addition of a Flag epitope permits manipulation or detection using anti-Flag antibodies and addition of a hexahistidine sequence permits manipulation or detection using Ni²⁺- or Co²⁺-affinity matrices. (See, for example, Robeva et al, 1996. Biochem. Pharmacol. 51, 545-555) Addition of both a Flag epitope and a hexahistidine sequence permits multiple applications, such as purification, immunoprecipitation and immunocytochemistry, which can aid in the development of assays to detect and/or quantify saxitoxin and/or saxitoxin derivatives in samples. Such assays are useful for analyzing samples, such as fish, crustaceans, mollusks, freshwater algae, water and tissues or fluids (e.g., blood, urine, saliva) from human, livestock and wildlife for saxitoxin and saxitoxin derivatives and, thus, detecting or aiding in the detection of paralytic shellfish poisoning.

The N-lobe of bullfrog saxiphilin has been shown to contain an insertion of two thyroglobulin type-1 domains which have been found to function as high-affinity inhibitors of cysteine proteases such as papain (B. Lenarcic, et al, 2000.). Papain inactivated by reagents such as iodoacetamide can thus be used to immobilize or affinity-purify saxiphilin using the native Thyr-1 domains which can be considered as native tag domains of the protein.

Tagged saxiphilin and tagged fragments thereof can be produced, using methods such as those exemplified herein (e.g., see the examples). In one embodiment, cells (e.g., insect cells) that contain a vector, such as a baculovirus vector, which comprises nucleic acids, (e.g., DNA) that encode tagged saxiphilin or tagged fragments thereof are cultured under conditions appropriate for expression of the encoded tagged saxiphilin or tagged fragments. For example, a vector comprising cDNA encoding saxiphilin and a Flag epitope, a hexahistidine sequence or both a Flag epitope and a hexahistidine sequence and additionally comprising DNA sufficient for expression in cells (e.g., transcriptional regulatory elements such as a promoter and an enhancer) can be introduced into appropriate host cells, which are cultured under conditions appropriate for expression of the encoded product (the encoded tagged saxiphilin), resulting in expression of tagged saxiphilin. In one embodiment, the vector is a baculovirus and the host cells are insect cells. Preferably, tagged saxiphilin is isolated from the cell culture, although it is possible to use a crude tagged saxiphilin-containing extract for detecting saxitoxin and/or saxitoxin derivatives. Tagged saxiphilin is isolated from cell culture using known methods, such as anti-flag antibodies or Ni²⁺-affinity or other metal chelate affinity matrices. The resulting tagged saxiphilin is substantially homogeneous (e.g., has been separated from other materials, such as components or constituents of cells in which it was produced). Tagged saxiphilin can include more than one Flag epitope and/or more than one hexahistidine sequence. In a specific embodiment, a vector comprising cDNA encoding bullfrog saxiphilin tagged with a Flag epitope, a hexahistidine sequence or both a Flag epitope and a hexahistidine sequence and additionally comprising DNA sufficient for expression in cells, such as insect cells, is introduced into host cells, which are cultured under conditions appropriate for expression of the encoded tagged bullfrog saxiphilin, resulting in production of bullfrog saxiphilin tagged with a Flag epitope, a hexahistidine sequence or Flag-hexahistidine (FH₆), respectively. Here, too, the tagged saxiphilin can comprise more than one Flag epitope and/or more than one hexahistidine sequence.

Similarly, tagged saxiphilin fragments that bind saxitoxin and saxitoxin derivatives can be produced. For example, a vector comprising cDNA encoding the C-lobe of saxiphilin (C-Sax cDNA) and a Flag epitope, a hexahistidine sequence or both a Flag epitope and a hexahistidine sequence and additionally comprising DNA sufficient for expression in cells is introduced into appropriate host cells, which are cultured under conditions appropriate for expression of the encoded product (the encoded tagged C-lobe of saxiphilin), resulting in expression of tagged C-lobe of saxiphilin (tagged C-Sax). Preferably, the resulting tagged saxiphilin fragments are isolated from the cell culture, although it is possible to use a crude tagged saxiphilin fragment-containing extract for detecting saxitoxin and saxitoxin derivatives. Tagged saxiphilin fragments are isolated from cell culture using known methods, such as anti-Flag antibodies or Ni²⁺- or other metal-chelate affinity matrices. The resulting tagged C-lobe of saxiphilin is substantially homogeneous (e.g., has been separated from other materials, such as components or consitutents of cells in which it was produced). In a specific embodiment, a vector comprising cDNA encoding bullfrog C-lobe saxiphilin tagged with a Flag epitope, a hexahistidine sequence or both a Flag epitope and a hexahistidine sequence and additionally comprising DNA sufficient for expression in cells (e.g., transcriptional regulatory elements such as a promoter and an enhancer), such as insect cells, is introduced into appropriate host cells, which are cultured under conditions appropriate for expression of the encoded tagged bullfrog C-lobe saxiphilin, resulting in production of C-lobe saxiphilin tagged with a Flag epitope, a hexahistidine sequence or Flag-hexahistidine (FH₆), respectively. Tagged saxiphilin fragments can comprise more than one Flag epitope and/or more than one hexahistidine. In a specific embodiment, a vector comprising cDNA encoding bullfrog C-lobe tagged with a Flag epitope and a hexahistidine (Flag-His₆-tagged bullfrog C-lobe saxiphilin) and additional sequences sufficient for expression of the encoded product is used, resulting in production of Flag-His₆-tagged bullfrog C-lobe saxiphilin (FH₆ C-lobe saxiphilin).

Tagged saxiphilin and tagged fragments are useful to detect STX or STX derivatives in a variety of formats. For example, a microtitre plate assay, in which tagged saxiphilin or tagged saxiphilin fragments are used, is one embodiment. Tagged saxiphilin or tagged saxiphilin fragments are placed into wells or attached to the surface of a microtitre plate, using known methods. Sample to be assessed (for presence or absence of saxitoxin or saxitoxin derivatives or their quantities) is contacted with tagged saxiphilin or tagged fragments thereof and binding (presence or absence, extent) of tagged saxiphilin or tagged fragments with saxitoxin or saxitoxin derivatives is determined, such as by detecting tagged saxiphilin-saxitoxin, tagged saxiphilin fragment-saxitoxin, tagged saxiphilin-saxitoxin derivative and/or tagged saxiphilin fragment-saxitoxin derivative complexes. Optionally, the extent of binding, and , thus, the quantity of saxitoxin and/or saxitoxin derivatives in a sample can be determined.

In a further embodiment, tagged saxiphilin or tagged fragments thereof can be used to allow physical immobilization of the protein in a test strip assay format with detection by a visual color change, or in a chip-based assay format with detection by surface plasmon resonance or other optical, electrical, or optical-electrical methods.

The amount of STX or STX derivative in a sample can optionally be measured within the wells of a microtitre plate. In one embodiment, the method of the present invention is a method of determining the amount of STX or STX derivative in a sample within the wells of a microtitre plate by carrying out the procedure described above and, further, determining the amount of STX or STX derivative in the sample by comparing the extent of binding in the sample to the extent of binding in an appropriate control. A control can be, for instance, a titration curve obtained by measuring binding of labeled STX to tagged saxiphilin or tagged saxiphilin fragments thereof in samples containing predetermined amounts of unlabeled saxitoxin or saxitoxin derivatives. Such a curve can be pre-established or can be produced simultaneously with assessment of samples.

Tagged saxiphilin and tagged fragments thereof can be used to prepare affinity reagents useful for separating, purifying or concentrating saxitoxin or saxitoxin derivatives. Such affinity reagents are useful, for example, in assessing water quality (e.g., to detect possible contamination by algal blooms). In one embodiment, tagged saxiphilin and/or a tagged fragment thereof is coupled or attached to a suitable solid support such as a flat surface, beads or a column. Beads bearing tagged saxiphilin and/or tagged fragments thereof can be packed in a cartridge or column. Coupling or attachment can be effected by a variety of methods, such as cyanogen bromide-activated matrices such as agarose; epoxy activated matrices; carboxymethylcellulose hydrazide; polyacrylamide hydrazide and oxirane acrylic beads. Saxitoxin and saxitoxin derivatives present in a sample that is contacted with/passed over a solid support bearing tagged saxiphilin and/or tagged fragments thereof become bound. Bound saxitoxin and derivatives can be eluted by known ureas (e.g., treatment with acid, urea or concentrated salts), resulting in at least preliminary separation from the samples. Assays can be carried out on this material or further separation can be performed.

In another embodiment, the method of the present invention is a method of isolating tagged saxiphilin or tagged saxiphilin fragments thereof, comprising concentrating the supernatant from host cells expressing and secreting tagged saxiphilin or a tagged saxiphilin fragment and subjecting the cell culture supernatant to affinity chromatography (e.g., metal-chelate affinity chromatography), whereby tagged saxiphilin or a tagged fragment thereof is isolated.

Optionally, further purification may be obtained. In another embodiment, the method of the present invention is a method of further isolating tagged saxiphilin or a tagged saxiphilin fragment thereof, comprising isolating tagged saxiphilin or a tagged fragment thereof by carrying out the procedure described above and further subjecting the isolated tagged saxiphilin or tagged saxiphilin fragment to Heparin Sepharose chromatography, whereby tagged saxiphilin or a tagged saxiphilin fragment thereof is further isolated.

In one embodiment, the present invention is a method of detecting saxitoxin (STX) and STX derivatives in a sample, comprising contacting a tagged saxiphilin or a tagged fragment thereof which binds STX or a STX derivative and a sample to be assessed under conditions appropriate for binding of tagged saxiphilin or a tagged fragment thereof with STX or a STX derivative, and determining whether binding of tagged saxiphilin or a tagged fragment thereof to STX or a STX derivative occurs, wherein if binding occurs, then STX or a STX derivative is detected in the sample.

Optionally, the amount of STX or STX derivative in a sample can be measured (quantified). In one embodiment, the method of the present invention is a method of determining the amount of STX or STX derivative in a sample by carrying out the procedure described above and, further, determining the extent to which binding occurs and comparing the extent of binding in the sample to the extent of binding in an appropriate control. A control can be, for instance, a titration curve obtained by measuring binding of tagged saxiphilin or tagged fragments thereof in samples containing predetermined amounts of saxitoxin or saxitoxin derivatives. Such a curve can be pre-established or can be produced simultaneously with assessment of samples.

A wide variety of samples can be assessed for saxitoxin and/or saxitoxin derivatives using methods and reagents of the present invention. For example, tissues or tissue extracts from fish or mammals that ingested contaminated material; invertebrates (molluscs, including shellfish and cephalopods); algae, cyanobacteria, dinoflagellates and bacteria can be assessed. Biological fluids such as blood, urine and saliva, and water samples can also be assessed.

The present invention also relates to a kit for detecting (determining the presence or absence of) saxitoxin or saxitoxin derivatives and/or measuring the quantity of saxitoxin or saxitoxin derivatives in samples. The kit comprises tagged saxiphilin and/or tagged saxiphilin fragments and labeled STX such as tritiated STX, biotin-labeled STX, and fluorophore-labeled STX. Optionally, the kit can include tagged saxiphilin and/or tagged saxiphilin fragments immobilized on a microtiter plate, bead, test strip or other solid matrix; a solution of a known amount of STX for positive control and calibration curves; and/or buffer solutions suitable for incubation and washing.

The present invention is illustrated by the following examples, which are not intended to be limiting in any way.

Materials and Methods

The Following Materials and Methods Were Used in the Following Examples.

Materials

SF9 and High Five™ insect cells, Grace's TNM-FH insect cell media, SF900 II SFM insect cell media, penicillin-streptomycin antibiotic mixture, X-gal, and FBS (fetal bovine serum) were from Gibco (Rockville, Md.). pBluebac4 vector, PCR II vector and the Bac-N-blue linear transfection kit, were from Invitrogen (Carlsbad, Calif.). Nickel nitrilo-triacetic acid (Ni²⁺-NTA) super-flow resin was from Qiagen (Valencia, Calif.). M2 anti-Flag monoclonal antibody and mouse anti-IgG were from Sigma (St Louis, Mo.). Poros20 Heparin Sepharose chromatography media and ConSep LC100 Fast Performance Liquid Chromatography system were from Millipore (Bradford, Mass.), Reacti-bind™ metal chelate plates were from Pierce (Rockford, Ill.). Filtron Ultracassette™ tangential flow dialysis apparatus was obtained from Pall Filtron (Northborough, Mass.). ECL detection kit was from Pierce (Rockford, Ill.) and SafeScint scintillation liquid fluid was from American Bioanalytical (Natick, Mass.). [11-³H]saxitoxin (24 Ci/mmol) was purchased from Amersham Life Science (Arlington Heights, Ill.).

### EXAMPLE 1

Construction of the Transfer Vector

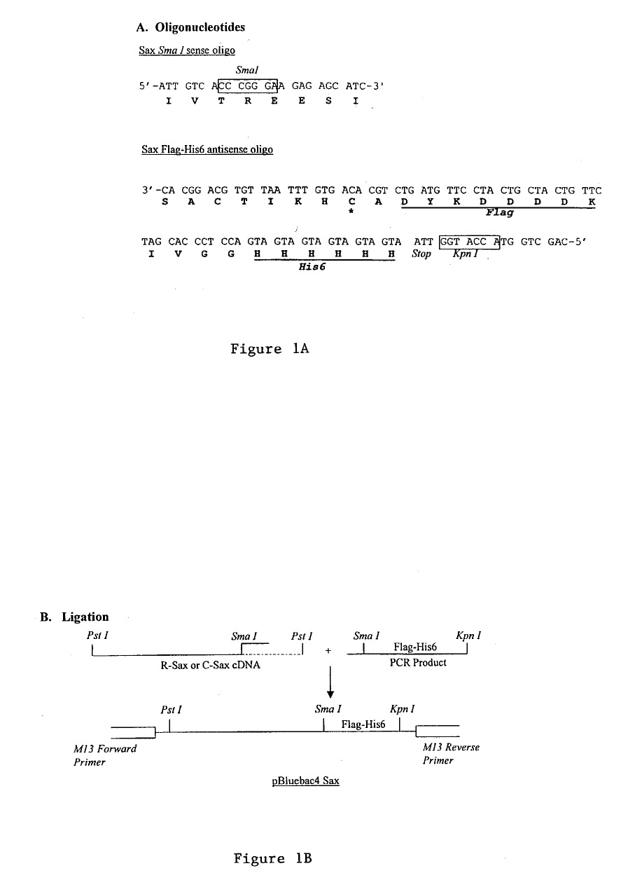

During the previous construction of C-Sax cDNA, a point mutation of Ser616Pro was inadvertently introduced by PCR (Morabito et al., 1995). This mutation was first corrected back to Ser616 by a standard site-specific mutagenesis procedure and confirmed by sequencing. A Flag-hexahistidine (FH₆) coding sequence was fused to R-Sax and C-Sax cDNA in pBluescript II KS⁻ as follows. An oligonucleotide primer overlapping the single SmaI restriction site near the C-terminal end of Sax cDNA and an antisense primer overlapping the position of the original stop codon of the Sax cDNA sequence (FIG. 1A) with the addition of the FH₆ tag extension, a new stop codon, and a new KpnI site were synthesized by the Yale nucleic acid facility. PCR amplification was performed using 1 μM primers and 0.1 μg R-Sax cDNA as the template, for 50 cycles (1 min at 94° C., 1 min at 42° C., 1.5 min at 72° C.). The resulting 350 basepair product known as the SmaI-KpnI FH₆ fragment was subcloned into PCRII, a cloning vector for PCR products.

To isolate the large fragment containing the entire R-Sax or C-Sax coding sequence, R-Sax and C-Sax cDNA in pBluescript II KS⁻ (Morabito et al., 1995) were first digested with PstI. This was followed by digestion of this fragment with SmaI. The resulting large PstI-SmaI (N-terminal) fragment was isolated and combined with the isolated SmaI-KpnI FH₆ (C-terminal) fragment described above. The two fragments were ligated into pBlueBac4 previously digested with PstI and KpnI to generate R-Sax-FH₆ and C-Sax-FH₆ constructs in pBluebac4 vector (FIG. 1B).

### EXAMPLE 2

Insect Cell Culture and Production of Recombinant R-Sax-FH₆ and C-Sax-FH₆

For virus production, SF9 insect (Spodoptera frugiperda) ovary cells were grown as adherent cells at 28° C. in Grace's TNM-FH medium supplemented with 10% FBS and penicillin and streptomycin (100 U/ml)). For protein production, High Five™ insect (Trichoplusia ni) cells were grown in serum free SF-900 II SFM media containing penicillin-streptomycin (100 U/ml), as suspension cultures with shaking at 150 rpm at 28° C. pBluebac 4 vector constructs of R-Sax-FH₆ and C-Sax-FH₆ were co-transfected with Bac-N-blue™ AcMNPV DNA into SF9 cells in the presence of cationic liposomes using the Bac-N-blue linearized transfection kit (Invitrogen) and incubated for 72 hours at 28° C. The supernatant from this co-transfection culture was diluted (1:10) and used in a plaque assay with agar containing X-gal to isolate pure recombinant plaques. After 7 days, blue plaques that were positive for lacZ, a marker for virus containing either R-Sax-FH₆ or C-Sax-FH₆, were picked from the plates. The recombinant virus was propagated in Grace's TNM-FH medium and purified by another round of plaque assay. Recombinant virus stocks were produced by amplification in SF9 cells, and assayed by the method of end-point dilution (Reilly et al., 1994).

For large scale protein production, High Five™ cells (2×10⁶/ml) were infected at a multiplicity of 5-10 pfu (plaque forming units) of virus per cell and grown at 28° C. with shaking at 150 rpm. The cell culture medium was harvested by centrifugation at 40 hours post-infection, supplemented with protease inhibitors (100 μg/ml phenylmethylsulfonylfluoride, 50 μg/ml leupeptin and 50 μg/ml pepstatin), and used for protein purification.

### EXAMPLE 3

[³H]STX Binding Assay

[³H]STX binding, Scatchard plot analysis, association and dissociation kinetics of [³H]STX binding to R-Sax-FH₆ and C-Sax-FH₆ were performed as described previously (Llewellyn and Moczydlowski, 1994). Binding isotherms for Scatchard analysis of R-SAX-FH₆ and C-SAX-FH₆ were obtained by varying the concentration of [³H]STX from 0.05 nM to 25 nM. Samples containing saxiphilin and [³H]STX were allowed to equilibrate on ice for 1-2 hours. Bound [³H]STX was separated from free toxin by rapid passage of 100 μl aliquots over mini columns of AG50W-X2 resin (100-200 mesh, Tris⁺, form from Bio-Rad, Hercules, Calif.), pre-equilibrated with 100 mM Tris-HCl, pH 7.4, 200 mM NaCl, and 10 mg/ml of bovine serum albumin. The standard incubation buffer for [³H]STX binding assay was 20 mM Mops-NaOH, pH 7.4 and 200 mM NaCl.

### EXAMPLE 4

SDS-PAGE and Immunoblot Analysis

Samples were subjected to SDS-PAGE (Laemmli, 1970) using a 10% acrylamide gel. The gel was blotted onto a nitrocellulose membrane using a Bio-Rad Mini Trans-Blot Cell. The membrane was incubated with 3% BSA in 20 mM Tris-HCl, 0.15 M NaCl, pH 7.5 (TBS) for 1 hour, and then incubated with 5 ml of a 10 μg/ml solution of M2 Anti-Flag antibody in TBS. After 3 washes with TBS (5 mins each) the membrane was incubated with a 1:500 dilution of horseradish peroxidase-conjugated anti-mouse IgG for 1 hour at 22° C. The membrane was once again washed 3 times with TBS, 5 mins each, and the protein bands were visualized by chemiluminescence detection (Pierce-ECL detection kit).

### EXAMPLE 5

Purification of R-Sax FH₆

One liter of baculovirus cell culture supernatant was concentrated to about 100 ml using a Filtron Ultracasette™ tangential flow dialysis apparatus and subsequently dialyzed against 4 liters of 150 mM NaCl, 50 mM sodium phosphate, pH 8.0 at 4° C., with at least two changes of solution. NaCl was added to a final concentration of 300 mM. 8 ml of Ni-NTA superflow resin was added to the cell supernatant and gently stirred for 8 hours on ice. The Ni-NTA resin containing bound R-Sax-FH₆ was packed in a column (2.0×15 cm) and washed with 10 volumes of buffer containing 50 mM sodium phosphate, 300 mM NaCl, pH 8.0, 30% glycerol, 4 mM imidazole. R-Sax-FH₆ was eluted by addition of 50 mM sodium phosphate, 300 mM NaCl, 150 mM imidazole pH 8.0 and active fractions were pooled.

The partially purified pool of R-Sax-FH₆ was then subjected to Heparin Sepharose chromatography using a Poros 20 Heparin Sepharose column on the ConSep LC100 Millipore system as follows. Poros 20 Heparin Sepharose media was packed and equilibrated in a 1×10 cm column according to the manufacturer's instructions. The sample of R-Sax-FH₆ was dialyzed against 4 litres of 10 mM sodium acetate, 10 mM MES buffer, 5 mM EDTA, pH 6.0 at 4° C. and loaded on the Poros 20 Heparin Sepharose column pre-equilibrated with dialysis buffer at a flow rate of 1 ml/min. The column was washed with 30 mM sodium acetate 10 mM MES, 5 mM EDTA at pH 6.0 and R-Sax-FH₆ was eluted with an increasing NaCl gradient (30-390 mM). The flow rate was 5 ml/min, with a gradient time of 14 min. Active [³H]STX-binding fractions were pooled, concentrated and analyzed by SDS-PAGE. Peak fractions were >90% pure as judged by silver or Coomassie blue protein staining. The specific activity of purified R-Sax-FH₆ was 6300 pmol [³H]STX binding sites per mg protein.

### EXAMPLE 6

Microtiter Well Assay of [³H]STX Binding and STX Competition

Reacti-bind™ Ni²⁺-chelate plates with separable wells were utilized in the following manner to assay saxiphilin-bound [³H]STX. A 200 μl reaction mixture containing 25 mM Hepes-NaOH, 150 mM NaCl, pH 8.0 was incubated with 5 nM [³H]STX per well. In the first type of experiment, [³H]STX bound to R-Sax-FH₆ was determined by incubating 5 nM [³H]STX with increasing amounts of R-Sax-FH₆. The reaction mix in the wells was incubated on ice for 3 hours and removed subsequently by aspiration. The wells were then washed three times with ice-cold 100 mM Tris-HCl, pH 8.0. Individual wells were then manually separated and placed into 25 ml liquid scintillation vials. [³H]STX was determined by liquid scintillation counting using 10 ml SafeScint liquid scintillation fluid. In the second type of experiment, a saturating concentration of R-Sax-FH₆ was incubated with 5 nM [³H]STX and titrated with increasing amounts of unlabeled STX, as a competitor in the binding reaction. [³H]STX bound in this experiment was plotted against the concentration of unlabeled STX in the reaction.

Results

Construction of Baculovirus Vectors, Virus Production and Protein Production

Expression of recombinant full-length saxiphilin (R-Sax) and C-lobe saxiphilin (C-Sax) in insect cells by use of baculovirus vectors has been described previously (Morabito et al., 1995). As described herein, these latter vectors were modified by addition of a cDNA sequence coding for 19 amino acids (ADYKDDDDKIVGGHHHHHH; SEQ ID NO: 17) at the original 3′ end. This cDNA sequence is: 5′ GCA GAC TAC AAG GAT GAC GAT GAC AAG ATC GTG GGA GGT CAT CAT CAT CAT CAT CAT 3′ (SEQ ID NO: 18). This modification was designed to introduce a Flag epitope (DYKDDDDK; SEQ ID NO: 7) followed by a hexahistidine sequence (HHHHHH; SEQ ID NO: 9) at the C-terminus of the expressed protein. The cDNA sequence that encodes the flag epitope is: 5′ GAC TAC AAG GAT GAC GAT GAC AAG 3′(SEQ ID NO: 8). The cDNA sequence that encodes the hexahistidine sequence is 5′ CAT CAT CAT CAT CAT CAT 3′ (SEQ ID NO: 10). FIG. 1A and 1B illustrate the construction procedure, which is described in further detail in the Methods section. Briefly, PCR was used to amplify a ˜350 base pair DNA fragment that overlapped an upstream 5′ SmaI restriction site and replaced the original stop codon with a synthetic extension containing the Flag-His₆ coding sequence and a new stop codon followed by a 3′ KpnI site. A ligation reaction with a PstI-SmaI fragment of the original R-Sax or C-Sax DNA was then used to construct a pBluebac4 vector for production of the respective baculovirus vectors. The coding regions of the modified portions of the final R-Sax-FH₆ and C-Sax-FH₆ constructions were fully sequenced to confirm that no errors were introduced.

Small scale stocks of R-Sax-FH₆ and C-Sax-FH₆ baculovirus were routinely produced by infecting SF9 cells with 0.1 pfu/cell and using this viral stock to produce the large-scale working stock for recombinant protein production. By the method of end-point dilution, the stock baculovirus titer for R-Sax-FH₆ and C-Sax-FH₆ was routinely found to be ˜7×10⁵ pfu/ml. High Five™ cells were utilized for recombinant protein production, since they secrete larger quantities of saxiphilin than SF9 cells. Baculovirus was added at a multiplicity of infection of 5 pfu/cell to one liter of High Five™ cells in mid-log phase. This culture was grown to a density of 2×10⁶ cells/ml at 25° C. with shaking at 150 rpm. After infection, aliquots of media were removed at various intervals and supernatant was prepared by centrifugation. [³H]STX binding activity of these samples was measured to assess the time course of protein production. The optimal level of secreted saxiphilin activity occurred at 40 hours post-transfection. Typical maximum levels of saxiphilin were 30 pmol/ml for R-Sax-FH₆ and 16.5 pmol/ml for C-Sax-FH₆ in the insect cell culture medium.

Purification of R-Sax-FH₆

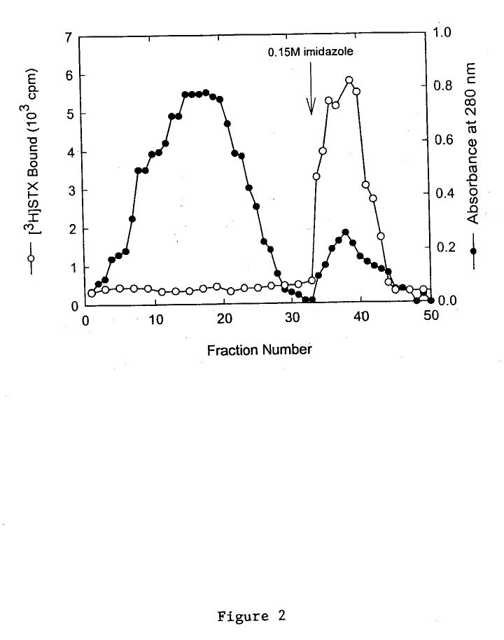

Previously, native bullfrog and untagged recombinant saxiphilin were routinely purified by Heparin Sepharose chromatography followed by chromatofocusing and Sepharose gel filtration (Li and Moczydlowski, 1991; Llewellyn and Moczydlowski, 1994). The purification of Flag-His₆-tagged proteins described here is simpler and faster than this latter procedure since it eliminates the laborious chromatofocusing step and subsequent gel filtration procedure required for ampholyte removal. FIG. 2 shows a typical fractionation of R-Sax-FH₆ from concentrated baculovirus media by Ni²⁺-NTA column chromatography. As this example illustrates, histidine-tagged proteins from crude mixtures can be readily purified by metal-chelate affinity chromatography using Ni²⁺-NTA resin. However this procedure can also result in contamination with extraneous proteins that contain histidine rich regions or that otherwise that bind to Ni²⁺-NTA. In the present work, several proteins from the insect cell media were found to co-purify with R-Sax-FH₆ on the Ni²⁺-NTA column. Hence, an additional step of Heparin Sepharose chromatography was employed for further purification. After this latter step, R-Sax-FH₆ was virtually homogeneous. The overall yield of this purification procedure was ˜20% of the original saxiphilin activity. The purified R-Sax-FH₆ preparation was also demonstrated to be homogenous by silver-staining. The molecular mass of R-Sax-FH₆ was ˜2 kD larger than untagged saxiphilin due to the presence of the 19-residue Flag-His₆ tag. Purified R-Sax-FH₆ was used in subsequent [³H]STX-binding experiments. C-Sax-FH₆ was partially purified using a single step of Ni²⁺-NTA affinity chromatography.

M2 anti-Flag monoclonal antibody was used to detect the Flag epitope on immunoblots. Both R-Sax-FH₆ and C-Sax-FH₆ were specifically recognized by M2 antibody as 93 and 40 kD bands, respectively.

[³H]STX Binding to Flag-His₆ Derivatives of Saxiphilin

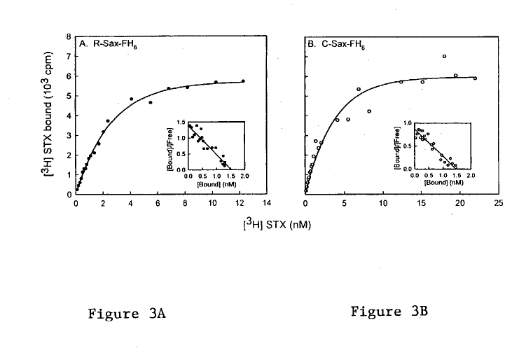

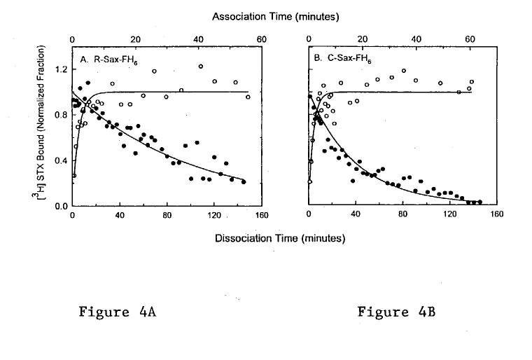

FIGS. 3A and 3B shows results of equilibrium binding titrations of R-Sax-FH₆ and C-Sax-FH₆ with increasing concentrations of [³H]STX. The insets are corresponding Scatchard plots for both proteins. For R-Sax-FH₆, K_{D} values were 1.0±0.08 nM and 0.5±0.03 nM from two separate experiments with a mean value of 0.75 nM. Corresponding K_{D} values for C-Sax-FH₆ binding were obtained as 1.58±0.15 nM, 2.07±0.2 nM and 4.5±0.45 nM from three separate experiments with a mean value of 2.7 nM. These K_{D} values for the Flag-His₆ tagged versions of saxiphilin and the C-lobe saxiphilin are approximately 3-fold higher than those measured for the corresponding untagged proteins (Morabito et al., 1995). The mean K_{D} for C-Sax-FH₆ is also approximately 3.5-fold higher than that of R-Sax-FH₆, similar to the 4-fold higher K_{D} previously observed for untagged C-Sax vs. R-Sax (Morabito et al., 1995). Measurements of the rates of [³H]STX association and dissociation are shown in FIGS. 4A and 4B. Bimolecular association rate constants were 8.1×10⁵ s⁻¹ M⁻¹ and 9.1×10⁵ s⁻¹ M⁻¹ for R-Sax-FH₆ and C-Sax-FH₆, respectively. The rate constant for dissociation of [³H]STX was measured by the rate of exchange of bound [³H]STX with a large excess of unlabeled STX. The dissociation time course was fit to a single exponential decay with rate constants of 1.7×10⁻⁴ s⁻¹ for R-Sax-FH₆ and 3.7×10⁻⁴ s⁻¹ for C-Sax-FH₆. The dissociation rate of the tagged C-lobe is about 2.5-fold faster than the corresponding tagged non-truncated version of saxiphilin. A similar difference was previously observed for untagged R-Sax and C-Sax (Morabito et al., 1995). K_{D} values calculated from the ratio of k_{off}/kₒₙ were 0.2 nM and 0.4 nM for R-Sax-FH₆ and C-Sax-FH₆, respectively. The source of discrepancy between the equilibrium and kinetic measurements of K_{D} is unknown at present. It may be related to the difficulty of achieving true equilbrium for high-affinity ligand binding reactions. In any case, it is clear that [³H]STX K_{D} values for both proteins are in the low nanomolar range. Microtitre plate assay of [³H]STX binding and competition by STX

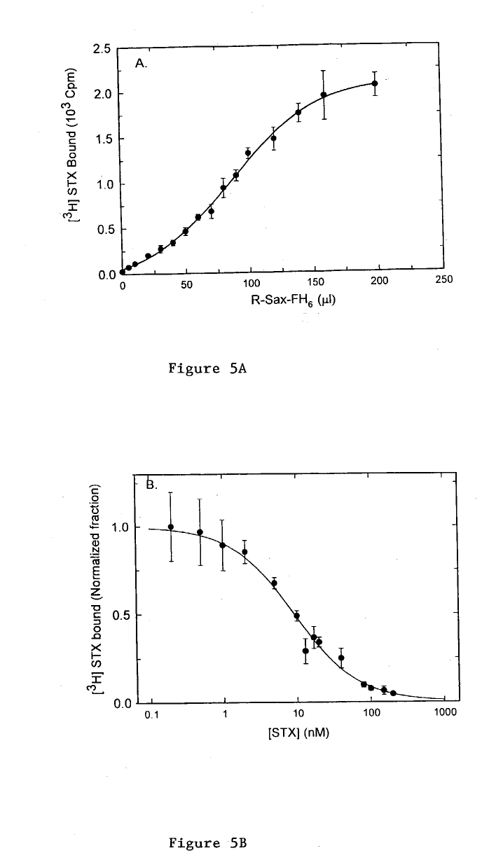

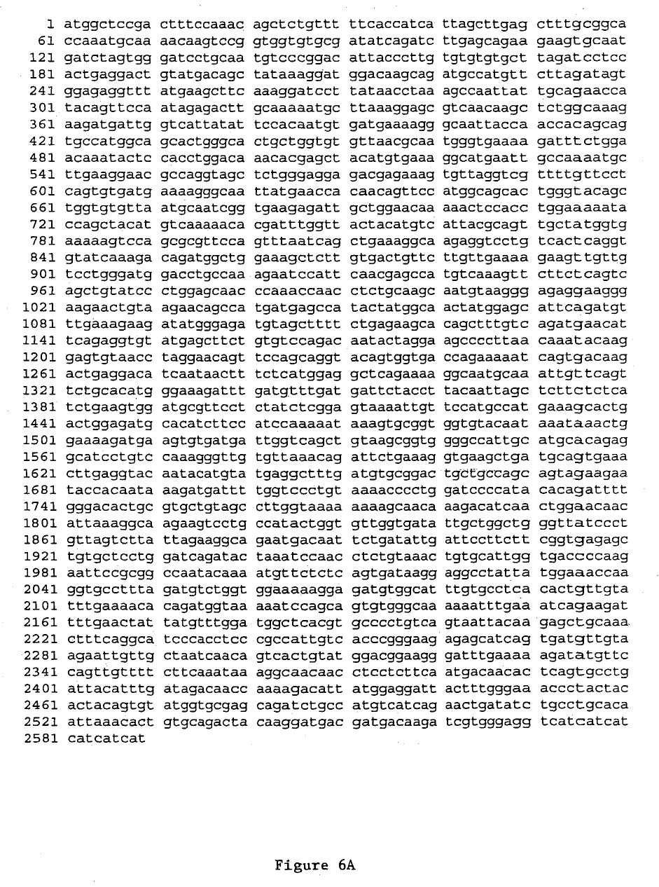

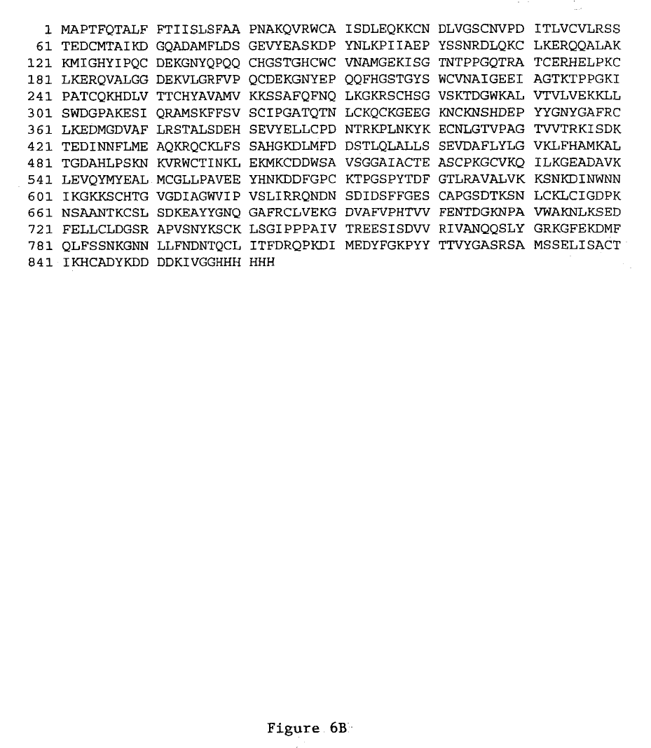

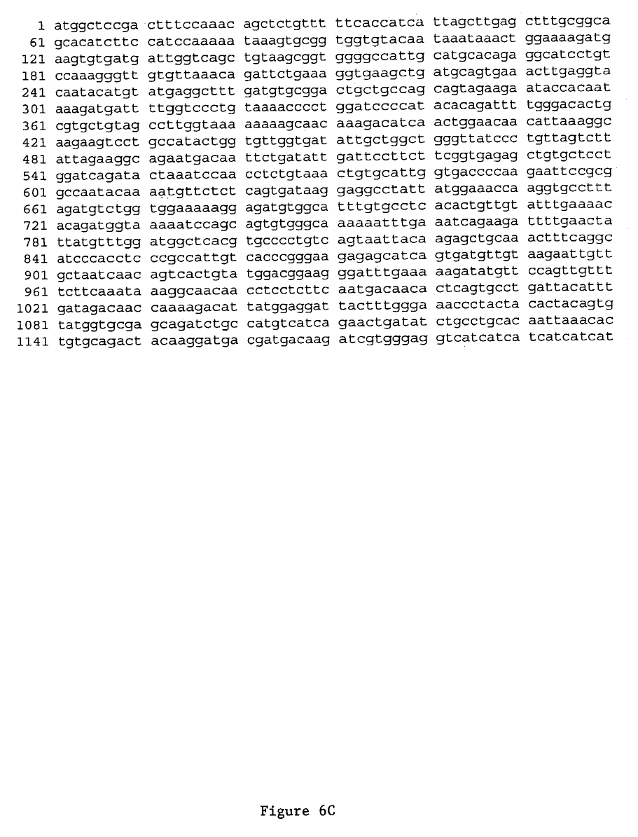

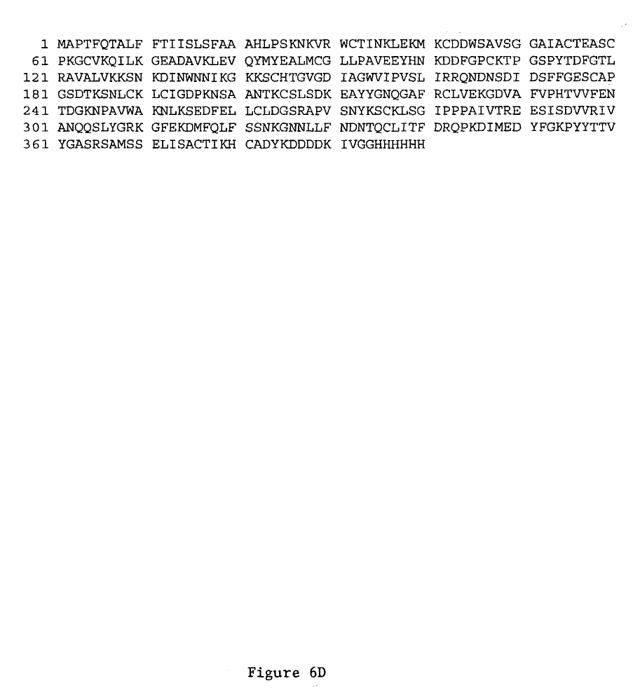

R-Sax-FH₆ binds to metal chelate affinity resins, such as the Ni²⁺-NTA resin and Reacti-Bind™ Ni²⁺-chelate plates, which are coated with the same functional groups. The binding of R-Sax-FH₆ to Ni²⁺-chelate plates exhibited a sigmoidal binding curve when titrated with an increasing amount of protein as shown in FIG. 5A. The amount of R-Sax-FH₆ bound to the Ni²⁺ chelate plates can be determined from radioactivity of [³H]STX bound to each well. Calculations indicate that each well is capable of binding ˜0.08 pmols of R-Sax-FH₆ at saturation. By using a constant amount of R-Sax-FH₆ and [³H]STX in each well, unlabeled STX added to the reaction mixture in the range of 0 to 200 nM was found to compete with [³ H]STX binding in a concentration-dependent fashion. From titrations such as FIG. 5B, the amount of unlabeled STX in an unknown reaction mixture can be thus be estimated by assay of STX standards and unknown samples under identical conditions. The background signal of this assay method was also found to be very low; e.g., about 50 cpm/well measured in the absence of R-Sax-FH₆ or in the presence of excess unlabelled STX. This corresponds to <2% of the total cpm/well bound in the presence of a saturating amount of R-Sax-FH₆ and 5 nM [³H]STX.

Discussion

This experiment describes the construction of baculovirus vectors for expression of Flag-hexahistidine tagged saxiphilin (R-Sax-FH₆) and tagged C-lobe saxiphilin (C-Sax-FH₆) in High Five™ insect cells. The Flag-hexahistidine coding sequence was placed as an insertion at the C-terminus of the protein so as not to interfere with protein folding and proper secretion.

The presence of the Flag-hexahistidine tag on these recombinant STX-binding proteins greatly facilitates their initial purification from crude insect cell culture medium as shown here for R-Sax-FH₆ using Ni²⁺-chelate affinity chromatography (FIG. 2). The Flag-epitope of the tagged proteins can also be exploited for immuno-affinity purification using commercially available anti-Flag antibodies covalently attached to a convenient chromatography matrix. As expected from the addition of a 19-residue tag, the R-Sax-FH₆ protein band migrates slightly slower than untagged recombinant saxiphilin on SDS-PAGE.

The kinetics of STX binding of R-Sax-FH₆ and C-Sax-FH₆ were compared to that of the untagged saxiphilins. The [³H]STX association rates of R-Sax-FH₆ and C-Sax-FH₆ were about 2-fold lower than the corresponding untagged versions of saxiphilin, which may be due to the presence of the Flag-hexahistidine tag sequence. However, the dissociation rate of [³H]STX from R-Sax-FH₆ is about the same as that of untagged saxiphilin. C-Sax-FH₆ exhibited a faster dissociation rate (3.7×10⁻⁴ s⁻¹) than that of R-Sax-FH₆ (1.7×10⁻⁴ s⁻¹), but slower than that of untagged C-lobe saxiphilin (7.0×10⁻⁴ s⁻¹) (Morabito et al., 1995). Despite these kinetic differences, the equilibrium binding affinity of the Flag-His₆ derivatives of saxiphilin for STX is in the nanomolar to subnanomolar range. The STX dissociation kinetics of these proteins are slow enough to permit a wide range of manipulations such as binding and concentration of STX from crude biological extracts and isolation of STX from tagged saxiphilin attached to solid matrices. In this regard, the pH-dependence of STX dissociation from saxiphilin (Llewellyn and Moczydlowski, 1994) can be utilized to elute free STX from saxiphilin at low pH.

R-Sax-FH₆ and C-Sax-FH₆ were recognized on a protein blot, by anti-Flag M2 antibodies. M2 antibody used in conjunction with secondary fluorescein-labeled IgG can be used to identify baculovirus-infected insect cells that secrete Flag-tagged saxiphilin by fluorescence microscopy. In addition, the hexahistidine tag on these proteins can be effectively utilized to immobilize STX-binding sites on Reacti-bind™ Ni⁺²-chelate microtiter plates, a procedure that can be adapted for a rapid quantitative assay for STX in algae or shellfish extracts. The initial application of this solid phase assay (FIGS. 5A and 5B) exhibited a low background of non-specific [³H]STX binding (<2%) and a sensitivity in the range of 1-50 nM corresponding to ˜20-80% displacement of 5 nM [³H]STX. In previous work, native bullfrog saxiphilin was shown to bind many other natural derivatives of STX with affinities ranging from 0.1 to 170 nM (Mahar et al., 1991). The solid-phase assay introduced here is useful for identifying neurotoxic samples collected in connection with dinoflagellate or cyanobacteria blooms. (See WO 02/48671 A1 Assay for Paralytic Shellfish Toxin, Table 1.)

Saxiphilin-like STX binding activity has also been identified in a number of distantly related animal species, such as Rana catesbiana (North American bullfrog), Bufo marinus (cane toad), Naja naja kaouthia (Thailand cobra), Thamnofis sirtalis (garter snake), Gambusia affnis (mosquito fish), and Ethmostigmus rubripes (Australian centipede) (Llewellyn et al., 1997). Although, only the cDNA sequence of saxiphilin from Rana catesbiana has so far been determined (Morabito and Moczydlowski, 1994a),. saxiphilins from diverse animal species may also be suitable for use in the methods, kits, and compositions of the invention.

Equivalents

As those skilled in the art will appreciate, numerous changes and modifications may be made to the preferred embodiments of the invention without departing from the spirit of the invention. It is intended that all such variations fall within the scope of the invention.

References

Anderson, D. M., 1989. Toxic algal blooms and red tides: a global perspective. In: Okaichi, T., Anderson, D. M., Nemoto T. (Ed.), Red Tides: Biology, Environment Science and Toxicology, pp. 11-21. New York: Elsevier

Baden, D. G., 1988. Public Health problems of red tides. In: Tu, A. T. (Ed.), Handbook of Natural toxins, Marine toxins and Venoms, pp. 259-277. New York: Marcel Dekker

Brinkman, A., Groffen, C., Kortleve, D. J., Geurts van Kessel, A., Drop, S. L., 1988. Isolation and characterization of a cDNA encoding the low molecular mass insulin-like growth factor binding protein (IBP-1). EMBO J. 7, 2417-2423.

Doucette, G. J., Logan, M. M., Ramsdell, J. S., Van Dolah, F. M., 1997. Development and preliminary validation of a microtiter plate-based receptor binding assay for paralytic shellfish poisoning toxins. Toxicon 35, 625-636.

Doyle, D. D., Wong, M., Tanaka, J., Barr, L., 1982. Saxitoxin binding sites in frog myocardial cytosol. Science 215, 1117-1119.

Durkin, M. E., Chakravarti, S., Bartos, B. B., Liu, S. H., Fridman, R. L., Chung, A. E., 1988. Amino acid sequence and domain structure of entactin. Homology with epidermal growth factor precursor and low density lipoprotein receptor. J. Cell. Biol. 107, 2749-2756.

Gessner, B. D., Bell, P., Doucette, G. J., Moczydlowski, E., Poli, M. A., Van Dolah, F., Hall, S., 1997. Hypertension and identification of a toxin in human urine and serum following a cluster of mussel-associated paralytic shellfish poisoning outbreaks. Toxicon 35, 711-722.

Indrasena, W. M., Gill, T. A., 1998. Fluorometric detection of paralytic shellfish poisoning toxins. Analytical Biochemistry 264, 230-236.

Koch, N., Lauer, W., Habicht, J., Dobberstein, B., 1987. Primary structure of the gene for murine Ia antigen-associated invariant chain (Ii). An alternately spliced exon encodes a cysteine-rich domain highly homologous to a repetitive sequence of thyroglobulin. EMBO J. 6, 1677-1683.

Kralovec, J. A., Laycock, M. V., Richards, R., Usleber, E., 1996. Immobilization of small molecules on solid matrices: A novel approach to enzyme-linked immunosorbent assay screening for saxitoxin and evaluation of anti-saxitoxin antibodies. Toxicon 34, 1127-1140.

Laemmli, U. K., 1970. Cleavage of structural proteins during the assembly of the head of bacteriophage T4. Nature (London) 227, 680-685.

Lenarcic, B., Ritonja, A., Strukelj, B., Turk, B., Turk, V., 1997. Equistatin, a new inhibitor of cysteine proteinases from Actinia equina, is structurally related to thyroglobulin type-1 domain. J. Biol. Chem. 272, 13899-13903.

Lenarcic, B, Krishnan, G, Borukhovich, R, Ruck, B, Turk, V and Moczydlowski, E, 2000. Saxiphilin, a saxitoxin-binding protein with two thyroglobulin Type 1 domains, is an inhibitor of papain-like cysteine proteinases. J. Biol. Chem. 275: 15572-15577.

Llewellyn, L. E., Doyle, J., Negri, A. P., 1998. A high-throughput, microtiter plate assay for paralytic shellfish poisons using the saxitoxin-specific receptor, saxiphilin. Anal. Biochem. 261, 51-56.

Li, Y., Moczydlowski, E. G., 1991. Purification and partial sequencing of saxiphilin, a saxitoxin-binding protein from the bullfrog, reveals homology to transferrin. J. Biol. Chem. 266, 15481-15487.

Llewellyn, L., Moczydlowski, E. G., 1994. Characterization of saxitoxin binding to saxiphilin, a relative of the transferrin family that displays pH-dependent ligand binding. Biochemistry 33, 12312-12322.

Llewellyn, L. E., Bell, P. M., Moczydlowski, E. G., 1997. Phylogenetic survey of soluble saxitoxin-binding activity in pursuit of the function and molecular evolution of saxiphilin, a relative of transferrin. Proc. R. Soc. Lond. B 264, 891-902.

Mahar, J., Lukas, G. L., Li, Y., Hall, S., Moczydlowski, E., 1991. Pharmacological and biochemical properties of saxiphilin, a soluble saxitoxin-binding protein from the bullfrog (Rana catesbiana). Toxicon 29, 53-71.

Moczydlowski, E. M., Mahar, J., Ravindran, A., 1988. Multiple saxitoxin-binding sites in bullfrog muscle: tetrodotoxin-sensitive sodium channels and tetrodotoxin-insensitive sites of unknown function. Mol. Pharmacology 33, 202-211.

Morabito, M. A., Llewellyn, L. E., Moczydlowski, E. G., 1995. Expression of saxiphilin in insect cells and localization of the saxitoxin-binding site to the C-terminal domain homologous to the C-lobe of transferrins. Biochemistry 34, 13027-13033.

Morabito, M. A., Moczydlowski, E., 1994a. Molecular cloning of bullfrog saxiphilin: A unique relative of the transferrin family that binds saxitoxin. Proc. Natl. Acad. Sci. USA 91, 2478-2482.

Morabito, M. A., Moczydlowski, E., 1994b. Molecular cloning of bullfrog saxiphilin. a unique relative of the transferrin familiy that binds saxitoxin. Proc. Natl. Acad. Sci. USA 92, 6651.

Negri, A., Llewellyn, L., 1998. Comparative analyses by HPLC and the sodium channel and saxiphillin ³H-Saxitoxin receptor assays for paralytic shellfish toxins in crustaceans and molluscs from tropical north west Australia. Toxicon 36, 283-298.

Robeva, A. S., Woodard, R., Luthin, D. R., Taylor, H. E., Linden, J., 1996. Double tagging recombinant A1 and A2A-adenosine receptors with hexahistidine and FLAG epitope. Development of an efficient generic protein purification procedure. Biochem Pharmacol. 51, 545-55.

Reilly, D., Miller, L. K., Luckow, V. A. 1994. Baculovirus expression vectors. In: D. Reilly, L. K. Miller, V. A. Luckow (Ed.), A laboratory manual, pp. 127-134. New York, Oxford: Oxford University Press

Schantz, E. J., McFarren, E. F., Schafer, M. L., Lewis, K. H., 1958. Purified shellfish poison for bioassay standardization. J. Ass. Off. Agric. Chem. 41, 160-168.

Shimizu, Y., 1996. Microalgal metabolites: a new perspective. Ann. Rev. Microbiol. 50, 431-465.

Sommer, H., Meyer, K. F., 1937. Paralytic shellfish poisoning. Arch. Path. 24, 560-598.

Sullivan, J J, Wekell, M M and Hall, S, 1988. “Detection of Paralytic Shellfish Toxins” in Handbook of Natural Toxins, vol. 3., 87-106 ed. A. T. Tu, Marcel Dekker, New York and Basel

We claim:

1. A method of detecting saxitoxin (STX) or STX derivatives in a sample, comprising:
(a) contacting a tagged saxiphilin or a tagged fragment thereof which binds STX or a STX derivative with a sample to be assessed under conditions appropriate for binding of tagged saxiphilin or a tagged fragment thereof with STX or a STX derivative, and
(b) determining whether binding of tagged saxiphilin or a tagged fragment thereof to STX or a STX derivative occurs in (a),
wherein if binding occurs, then STX or a STX derivative is detected in the sample.

2. The method of claim 1, wherein the tagged saxiphilin or tagged fragment thereof which binds STX or a STX derivative comprises a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (t) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

3. The method of claim 2, wherein the tagged saxiphilin or tagged fragment thereof which binds STX or a STX derivative comprises a tag selected from the group consisting of: (a) a Flag epitope; (b) a hexahistidine sequence; and (c) a Flag epitope and hexahistidine sequence.

4. The method of claim 1, wherein the amount of STX or a STX derivative in the sample is measured, further comprising determining the extent to which binding of tagged saxiphilin or a tagged fragment thereof with STX or a STX derivative occurs and comparing the extent of binding that occurs in the sample to the extent of binding in an appropriate control.

5. The method of claim 4, wherein the tagged saxiphilin or tagged fragment thereof which binds STX or a STX derivative comprises a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

6. The method of claim 5, wherein the tagged saxiphilin or tagged fragment thereof which binds STX or a STX derivative comprises a tag selected from the group consisting of: (a) a Flag epitope; (b) a hexahistidine sequence; and (c) a Flag epitope and hexahistidine sequence.

7. A method of detecting STX in a sample, comprising:
(a) combining a saturating concentration of tagged saxiphilin or a tagged fragment thereof which binds STX or a STX derivative bound to a solid support with labeled STX and increasing amounts of a sample containing unlabelled STX;
(b) measuring the amount of labeled STX bound to the solid support; and
(c) determining the amount of unlabelled STX in the sample,
wherein STX is detected in the sample by comparing the amount of labeled STX bound to the solid support with the amount of labeled STX bound to the solid support in an appropriate control.

8. A method of detecting STX in a sample, comprising:
(a) combining a saturating concentration of tagged saxiphilin or a tagged fragment thereof which binds STX or a STX derivative with labeled STX and increasing amounts of a sample containing unlabelled STX within the wells of a metal-chelate microtitre plate;
(b) measuring the amount of labeled STX bound to each well; and
(c) determining the amount of unlabelled STX in the sample,
wherein STX is detected in the sample by comparing the amount of labeled STX bound to each well with the amount of labeled STX bound to each well in an appropriate control.

9. The method of claim 7, wherein the tagged saxiphilin or tagged fragment thereof which binds STX or a STX derivative comprises a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

10. The method of claim 9, wherein the tagged saxiphilin or tagged fragment thereof which binds STX or a STX derivative comprises a tag selected from the group consisting of: (a) a Flag epitope; (b) a hexahistidine sequence; and (c) a Flag epitope and hexahistidine sequence.

11. The method of claim 7, wherein the solid support is a microtitre plate, bead or test strip.

12. The method of claim 11, wherein the solid support is selected from the group consisting of cyanogen bromide-activated matrices such as agarose; epoxy activated matrices; carboxymethylcellulose hydrazide; polyacrylamide hydrazide and oxirane acrylic beads.

13. The method of claim 7, wherein the labeled STX is [³H]STX, biotin-labeled STX or fluorophore-labeled STX.

14. The method of claim 7, wherein the amount of STX in the sample is measured, further comprising comparing the amount of labeled STX combined with the sample with the amount of labeled STX combined with an appropriate control comprising known amounts of unlabeled STX.

15. Tagged saxiphilin or a tagged fragment thereof which binds STX or a STX derivative, comprising saxiphilin or a fragment thereof, and a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

16. Tagged saxiphilin or a tagged fragment thereof of claim 15, wherein the tagged saxiphilin or tagged fragment thereof comprises saxiphilin or a fragment thereof, and a tag selected from the group consisting of: (a) a Flag epitope; (b) a hexahistidine sequence; and (c) a Flag epitope and a hexahistidine sequence.

17. The tagged saxiphilin or tagged fragment thereof of claim 15, wherein the Flag epitope comprises an amino acid sequence of SEQ ID NO: 7.

18. The tagged saxiphilin or tagged fragment thereof of claim 15, wherein the tag is present at the C-terminus of saxiphilin or a fragment thereof.

19. The tagged saxiphilin protein of claim 15 wherein the saxiphilin is derived from bullfrog.

20. The tagged saxiphilin protein of claim 15 wherein the saxiphilin is derived from Rana catesbiana.

21. Tagged saxiphilin comprising the amino acid sequence of SEQ ID NO: 12 or SEQ ID NO: 14.

22. Isolated nucleic acid comprising a nucleic acid sequence that encodes a tagged saxiphilin or a tagged fragment thereof which binds STX or a STX derivative, comprising saxiphilin or a fragment thereof, and a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

23. Isolated nucleic acid of claim 22, wherein the tag is selected from the group consisting of: (a) a Flag epitope; (b) a hexahistidine sequence; and (c) a Flag epitope and a hexahistidine sequence.

24. Isolated nucleic acid of claim 22, wherein the Flag epitope comprises the nucleic acid sequence of SEQ ID NO: 8.

25. Isolated nucleic acid of claim 22, wherein the tag is present at the C-terminus of saxiphilin or a fragment thereof.

26. Isolated nucleic acid comprising the nucleic acid sequence of SEQ ID NO: 11 or SEQ ID NO: 13.

27. A vector comprising a nucleic acid encoding tagged saxiphilin or a tagged fragment thereof which binds STX or a STX derivative, wherein the tagged saxiphilin or tagged fragment thereof comprises a tag selected from the group consisting of: (a) a Flag epitope; (b) a hexahistidine sequence; (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

28. The vector of claim 27 wherein the tag is selected from the group consisting of: (a) a Flag epitope; (b) a hexahistidine sequence; and (c) a Flag epitope and a hexahistidine sequence.

29. The vector of claim 27, wherein the nucleic acid encoding the tagged saxiphilin comprises the nucleic acid sequence of SEQ ID NO: 11 or SEQ ID NO: 13.

30. The vector of claim 27, wherein the tag is present at the C-terminus of saxiphilin or fragment thereof.

31. The vector of claim 27 additionally comprising nucleic acid sufficient for expression of the nucleic acid encoding tagged saxiphilin or a tagged fragment thereof.

32. Host cells comprising an exogenous nucleic acid encoding tagged saxiphilin or a tagged fragment thereof which binds STX or a STX derivative, wherein the tagged saxiphilin or tagged fragment thereof comprises a tag selected from the group consisting of: (a) a Flag epitope; (b) a hexahistidine sequence; and (c) a streptavidin sequence (Strep-tag); (d) calmodulin binding peptide (CBP); (e) maltose binding protein (MBP); (f) cellulose binding domain (CBD) tag; (g) myc epitope; (h) hemmaglutinin (HA) tag; (i) glutathione-S-transferase (GST) fusion; and (j) thioredoxin fusion, and combinations of any of (a)-(j).

33. Host cells of claim 32, wherein the tag is selected from the group consisting of: (a) a Flag epitope; (b) a hexahistidine sequence; and (c) a Flag epitope and a hexahistidine sequence.

34. The host cells of claim 32, wherein the exogenous nucleic acid is a vector.

35. Host cells comprising the vector of claim 27, wherein the nucleic acid encoding the tagged saxiphilin comprises the nucleic acid sequence of SEQ ID NO: 11 or SEQ ID NO:13.

36. Host cells comprising the vector of claim 27, wherein the tag is present at the C-terminus of saxiphilin or fragment thereof.

37. Host cells comprising the vector of claim 27, additionally comprising nucleic acid sufficient for expression of the nucleic acid encoding tagged saxiphilin or a tagged fragment thereof.

38. Method of producing tagged saxiphilin or a tagged fragment thereof comprising: culturing cells that contain nucleic acid encoding tagged saxiphilin or a tagged fragment thereof under conditions appropriate for expression of the nucleic acid, thereby producing tagged saxiphilin or a tagged saxiphilin fragment.

39. The method of claim 38, further comprising isolating the tagged saxiphilin or tagged saxiphilin fragment, thereby producing isolated tagged saxiphilin or tagged saxiphilin fragment.

40. The method of claim 39, wherein isolating the tagged saxiphilin or tagged saxiphilin fragment comprises:
(a) concentrating the supernatant from host cells expressing and secreting tagged saxiphilin or a tagged saxiphilin fragment; and
(b) subjecting the cell culture supernatant to affinity chromatography,
wherein tagged saxiphilin or a tagged saxiphilin fragment is thereby isolated.

41. The method of claim 40 further comprising subjecting the isolated tagged saxiphilin or tagged saxiphilin fragment to Heparin Sepharose chromatography, wherein the tagged saxiphilin or tagged saxiphilin fragment is thereby further isolated.

42. A kit for detecting the amount of saxitoxin or saxitoxin derivatives in a sample comprising:
(a) tagged saxiphilin or tagged saxiphilin fragment; and
(b) labeled STX or labeled STX derivative.

43. A kit for detecting the amount of saxitoxin or saxitoxin derivatives in a sample comprising:
(a) tagged saxiphilin or tagged saxiphilin fragment immobilized on a solid support; and
(b) labeled STX or labeled STX derivative.

44. The kit of claim 43, wherein the solid support is a microtiter plate, bead or test strip.

45. The kit of claim 43, wherein the solid support is selected from the group consisting of cyanogen bromide-activated matrices such as agarose; epoxy activated matrices; carboxymethylcellulose hydrazide; polyacrylamide hydrazide and oxirane acrylic beads.

46. The kit of claim 42 or 43, wherein the labeled STX or labeled STX derivative is [³H]STX, biotin-labeled STX or fluorophore-labeled STX.

47. The kit of claim 42 or 43, further comprising buffer solutions suitable for incubation and washing.

48. A kit for measuring the amount of saxitoxin or saxitoxin derivatives in a sample comprising:
(a) tagged saxiphilin or tagged saxiphilin fragment; and
(b) labeled STX or labeled STX derivative.

49. A kit for measuring the amount of saxitoxin or saxitoxin derivatives in a sample comprising:
(a) tagged saxiphilin or tagged saxiphilin fragment immobilized on a solid support; and
(b) labeled STX or labeled STX derivative.

50. The kit of claim 49, wherein the solid support is a microtiter plate, bead or test strip.

51. The kit of claim 49, wherein the solid support is selected from the group consisting of cyanogen bromide-activated matrices such as agarose; epoxy activated matrices; carboxymethylcellulose hydrazide; polyacrylamide hydrazide and oxirane acrylic beads.

52. The kit of claim 48 or 49, wherein the labeled STX or labeled STX derivative is [³H]STX, biotin-labeled STX or fluorophore-labeled STX.

53. The kit of claim 48 or 49, further comprising a solution of a known amount of STX for calibration.

54. The kit of claim 48 or 49, further comprising buffer solutions suitable for incubation and washing.
